(19) United States
(12) Patent Application Publication
(10) Pub. No.: US 2017/0348121 A1
(43) Pub. Date: Dec. 7, 2017

(54) STENT AND METHOD OF MANUFACTURING STENT

(71) Applicant: TERUMO KABUSHIKI KAISHA, Tokyo (JP)

(72) Inventor: Toshihiro YAMAMOTO, Ashigarakami-gun (JP)

(73) Assignee: TERUMO KABUSHIKI KAISHA, Tokyo (JP)

(21) Appl. No.: 15/682,972

(22) Filed: Aug. 22, 2017

Related U.S. Application Data
(63) Continuation of application No. PCT/JP2016/052543, filed on Jan. 28, 2016.

(30) Foreign Application Priority Data
Feb. 27, 2015 (JP) 2015-039349

(51) Int. Cl. A61F 2/89; B29C 65/02; B29L 31/00

(52) U.S. Cl. CPC A61F 2/89; B29C 65/02; A61F 2230/0069; A61F 2240/00; B29L 2031/753

(57) ABSTRACT

A stent and a stent manufacturing method results in a stent exhibiting improved fracture resistance. The stent has metal portions that shape a tubular outer periphery provided with a gap and a polymer portion that connects the metal portions to each other in the gap. The polymer portion has a curved portion that is curved to be concave toward the outer side from the inner side in the radial direction of the tubular outer periphery.

### CROSS-REFERENCES TO RELATED APPLICATIONS

This application is a continuation of International Application No. PCT/JP2016/052543 filed on Jan. 28, 2016, and claims priority to Japanese Application No. 2015-039349 filed on Feb. 27, 2015, the entire content of both of which is incorporated herein by reference.

### TECHNICAL FIELD

The present invention generally relates to a stent and a method of manufacturing a stent.

### BACKGROUND DISCUSSION

In recent years, a technique of forming a stent using metal and polymer has been proposed. For example, International Application Publication No. 2007/079363 discloses a stent in which helical outer peripheral portions are formed of metal, and a connection portion that connects the helical metal portions to each other is formed of polymer. By forming the stent using metal and polymer, it is possible for the stent to exhibit the contradictory properties of strength and flexibility.

### SUMMARY

However, when a tensile force is applied to an interface between metal and polymer such that the metal and the polymer are separated from each other, fracture resistance is reduced compared to a case where the entire stent is integrally formed of only metal or only polymer. The inventor here has discovered that the fracture resistance of the stent can be improved by hindering a strong force from being applied to the interface between the metal and the polymer.

The stent exhibits improved fracture resistance and the manufacturing method results in a stent exhibiting such characteristics.

According to one aspect, a stent comprises metal portions that together form a tubular frame possessing an outer periphery, with the tubular frame including a gap extending through the tubular frame and at which two of the metal portions are positioned adjacent one another in a spaced-apart manner; and a polymer portion located in the gap and connecting the two metal portions to each other. The polymer portion includes a curved portion that is curved and possesses a concave shape that is recessed toward an outer side of the stent from an inner side of the stent in a radial direction of the outer periphery.

According to another aspect, a stent comprises: metal portions that together form a tubular frame possessing an outer periphery, wherein the tubular frame includes a gap extending through the tubular frame and at which two of the metal portions are positioned adjacent one another in a spaced-apart manner; and a polymer portion formed of biodegradable polymer and connecting the two metal portions to each other. The polymer portion possesses an inwardly facing side facing towards an interior of the frame, the inwardly facing side of the polymer portion being curved.

Another aspect involves a method of manufacturing a stent, comprising: placing polymer in contact with two metal portions of a tubular stent frame that possesses an outer periphery, with the two metal portions being spaced apart from one another so that a gap exists between the two metal portions; and heating the polymer after placing the polymer in contact with the two metal portions to connect together the two metal portions by way of the polymer portion. The heating comprising heating the polymer so that the polymer is molten and flows to the gap to form a polymer portion that connects the two metal portions and includes an inwardly facing curved portion that is curved and possesses a concave shape that is recessed toward an outer side of the stent from an inner side of the stent in a radial direction of the outer periphery.

The polymer portion which is relatively easily stretchable compared to the metal portions is thinned by forming the curved portion. Therefore, the polymer portion becomes more easily stretchable. For this reason, when a tensile force is applied to separate the metal portions and the polymer portion from each other, a strong force is not easily applied to an interface between the metal portions and the polymer portion by virtue of the stretch of the polymer portion. Therefore, it is possible to more improve the fracture resistance.

### BRIEF DESCRIPTION OF THE DRAWINGS

FIG. 1 is a perspective view of a stent according to an embodiment representing an example of the inventive step disclosed here.

FIG. 2 is a cross-sectional view taken along the section line 2-2 of FIG. 1.

FIGS. 3A-3C are diagrams illustrating an overview of a method of manufacturing a stent according to an embodiment.

FIG. 4 is a cross-sectional view illustrating a modification of a polymer portion.

FIG. 5 is a cross-sectional view illustrating another modification of the polymer portion.

FIG. 6 is a cross-sectional view illustrating a modification in which a polymer layer is formed along with the polymer portion.

FIG. 7 is a cross-sectional view illustrating another modification in which a polymer layer is formed along with the polymer portion.

FIG. 8 is a cross-sectional view illustrating further another modification of the polymer portion.

FIG. 9 is an enlarged view illustrating a main portion according to a modification in which a connection portion is provided along with the polymer portion.

FIG. 10 is a cross-sectional view taken along the section line 10-10 of FIG. 9.

### DETAILED DESCRIPTION

Set forth below with reference to the accompanying drawings is a detailed description of embodiments of a stent and a stent manufacturing method representing examples of the inventive stent and manufacturing method disclosed here. The dimensions or scales on the drawings may be exaggerated or different from actuality/reality for convenience of description and illustration.

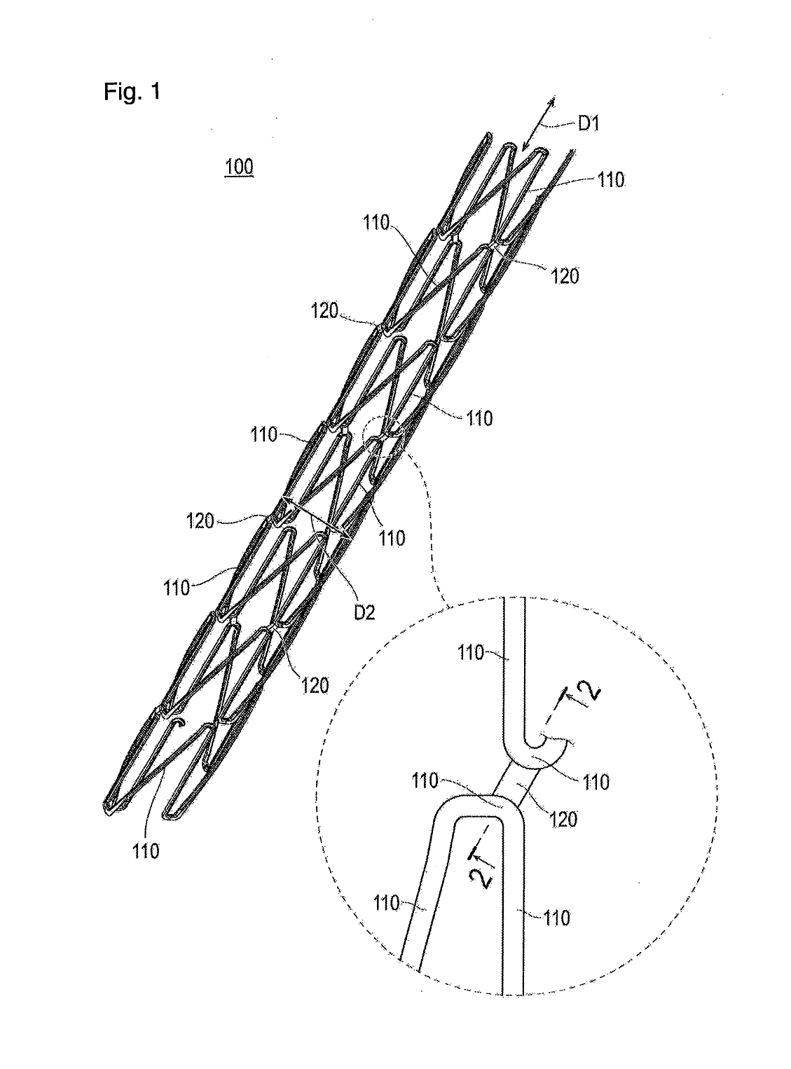

As illustrated in FIG. 1, a stent 100 according to an embodiment has a plurality of struts 110 (metal portion) and a plurality of polymer portions 120.

The stent 100 is used in or positionable in a lumen such as a blood vessel, a bile duct, a trachea, an esophagus, a gastrointestinal tract, and a urethra in a living body. The stent 100 treats stenosis or obstruction by forcibly widening or enlarging the lumen. The stent 100 may be a balloon-expandable stent which is expanded by inflating a balloon (i.e., the stent surrounds a balloon and inflation/expansion of the balloon expands the stent) or a self-expandable stent which expands by its own expanding function.

The struts 110 include linear components formed of metal as well as curved components formed of metal that interconnect the linear components as shown in the enlarged portion of FIG. 1. The integrated struts 110 are shaped or configured to define a tubular member or tubular frame having an outer periphery with gaps in the tubular frame. In the expanded state, the tubular frame defined by the metal portions or metal struts is a cylindrical frame.

For example, the struts 110 may be connected and arranged to form a wavy-shaped member, with axially oriented (axially extending) peaks and valleys as shown in FIG. 1, that extends helically in the axial direction D1 of the stent 100 to form an endless annular body (endless from one axial end of the annular body/stent to the opposite axial end of the annular body/stent). In addition, some of the struts 110 are connected coaxially along the axial direction D1 of the stent 100, by the polymer portions 120, so as to shape the outer periphery of the stent 100. Alternatively, the struts 110 may be interconnected and arranged to form a plurality of wavy-shaped endless annular members that are axially arranged along the axial extent of the stent, with axially adjacent wavy-shaped endless annular members connected coaxially in the axial direction D1 of the stent 100, by the polymer portions 120, so as to shape the outer periphery of the stent 100. The shape of the struts 110 is not particularly limited. The axial direction D1 of the stent 100 is perpendicular to a radial direction D2 of the tubular outer periphery of the stent 100 (hereinafter, simply referred to as a radial direction D2 of the stent 100).

The metal forming the struts 110 may include, for example, stainless steel, tantalum, tantalum alloy, titanium, titanium alloy, nickel titanium alloy, tantalum titanium alloy, nickel aluminum alloy, Inconel, gold, platinum, iridium, tungsten, tungsten alloy, cobalt-based alloy such as cobalt chromium alloy, magnesium, zirconium, niobium, zinc, or silicon, but not particularly limited thereto. The metal from which the struts 110 are fabricated may be either biodegradable metal or non-biodegradable metal.

Each of the polymer portions 120 is positioned in a gap between two adjacent struts 110 to connect the struts 110 to each other. In the illustrated embodiment, the polymer portions 120 connect together spaced apart (axially spaced apart) and adjacent struts (axially adjacent struts) 110. The polymer portions 120 are located in the gaps of openings in the stent (i.e., the gaps/openings that communicate the interior of the stent with the exterior of the stent). There is no particular limitation to where the polymer portions 120 are provided or located in the gap of the outer periphery of the stent 100 as long as the polymer portions 120 connect metal members of the stent 100 to each other.

The polymer portions 120 are formed of, for example, biodegradable polymer. The biodegradable polymer includes, for example, a biodegradable synthetic polymer material polylactic acid, polyglycolic acid, lactic acid-glycolic acid copolymer, polycaprolactone, lactic acid-caprolactone copolymer, glycolic acid-caprolactone copolymer, poly-γ-glutamin acid, a natural biodegradable polymer material such as cellulose or collagen, or the like. The polymer portions 120 may also be formed of non-biodegradable polymer.

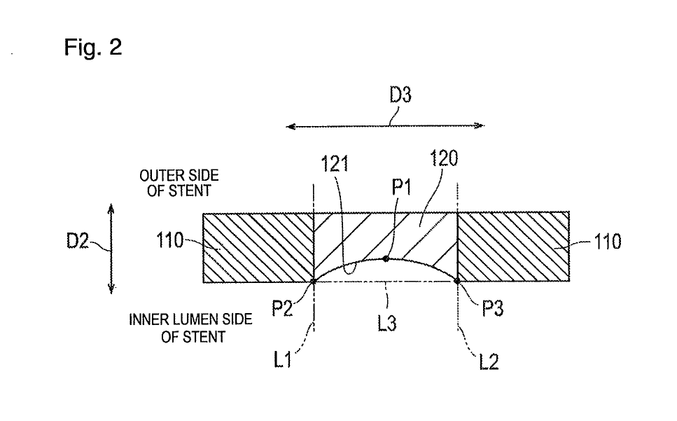

As illustrated in FIG. 2, each of the polymer portions 120 has a curved portion 121 in an inner lumen side (inner side) of the stent 100. That is, the polymer portions 120 are positioned on the inner side of the stent that faces toward the interior of the stent. The curved portion 121 is curved to be concave from the inner lumen side toward the outer side in the radial direction D2 of the stent 100. The curved portion 121 has a curvature different from the circumferential curvature of the stent 100. The circumferential curvature of the stent 100 refers to the radius of curvature of the inner surface of the stent when the stent is expanded.

In the cross section of FIG. 2 (a cross section taken along the separation direction of the metal portions), a peak P1 where the curved portion (concavity) 121 is deepest is located in a center between two boundary lines L1 and L2 formed between the polymer portion 120 and two adjacent struts 110. That is, the polymer portion 120 is connected to one of the two metal struts 110 at one boundary L1 and the polymer portion is connected to the other of the two metal struts 110 at the other boundary L2, and the most recessed point of the concave-shaped curved portion 121 (i.e., the peak P1 of the concave-shaped curved portion 121) is located at the center between the two boundaries L1, L2. In addition, the peak P1 is deviated from a line L3 obtained by connecting, with a straight line, two cross points P2 and P3 between the boundary lines L1 and L2 and the contour line of the curved portion 121. That is, the peak P1 exists in a different position that does not cross the line L3 obtained by connecting, with a straight line, the two cross points P2 and P3 between the boundary lines L1 and L2 and the contour line of the curved portion 121. The peak P1 is thus spaced from the line L3.

Next, a method of manufacturing the stent 100 will be described.

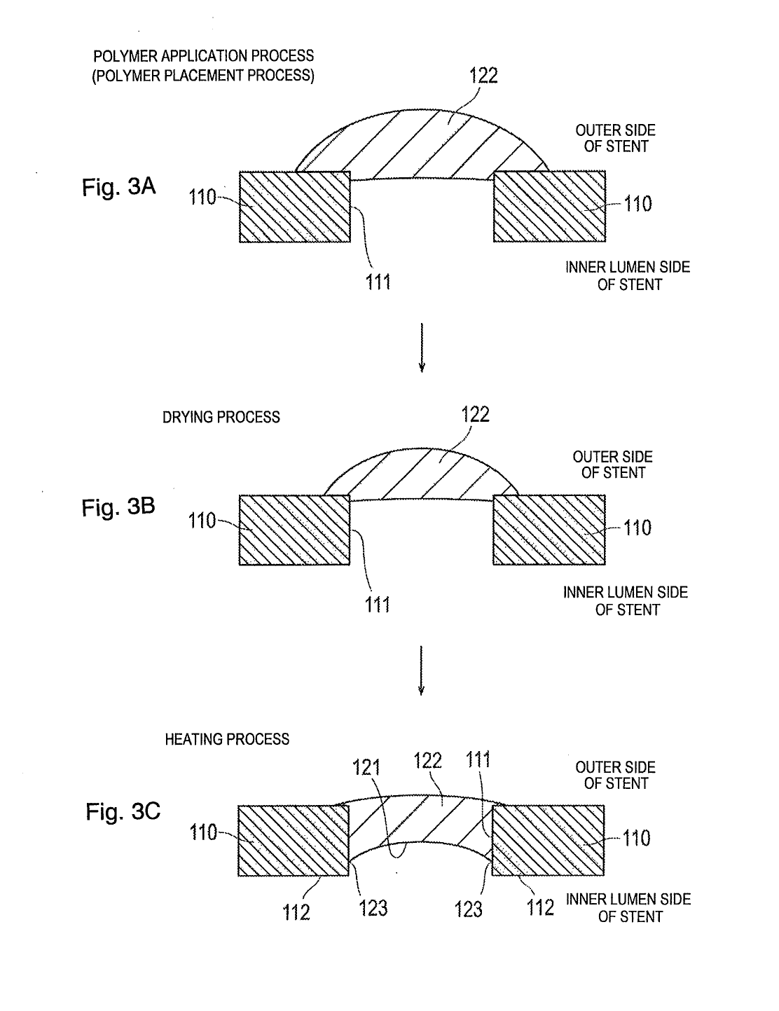

As illustrated in FIGS. 3A-3C, the method of manufacturing the stent 100 includes a polymer application process, a drying process, and a heating process. Before the polymer application process, the frame defined by the interconnected or integrated struts 110 and having a predetermined shape or configuration is prepared.

In the polymer application process, a polymer solution 122 is applied toward the gap 111 formed by the adjacent struts 110. The polymer solution 122 is applied toward the gap 111 from the outer side of the stent (i.e., the side facing outwardly away from the stent interior). The polymer solution 122 is applied using an application device such as a micro-syringe.

The polymer solution 122 is obtained by dissolving polymer of the polymer portions 120 in a solvent. The solvent includes, for example, an organic solvent such as methanol, ethanol, dioxane, tetrahydrofuran, dimethylformamide, acetonitrile, dimethylsulfoxide, or acetone, and the like.

In the drying process after the polymer application process, the polymer solution 122 applied to the struts 110 is dried, and the solvent is evaporated. The drying of the polymer solution 122 may include, for example, natural drying. Alternatively, the drying may include heated drying by heating the polymer solution 122. The heated drying is not limited to a particular type of heated drying. The drying reduces the volume of the polymer solution 122 and increases the viscosity of the polymer solution 122.

After the drying process, the dried polymer solution 122 is heated in the heating process to further evaporate the solvent and melt the polymer contained in the solution. In the heating process, for example, the polymer solution 122, attached to the struts 110, is heated inside a vacuum furnace together with the struts 110. Here, the heating temperature of the polymer solution 122 may be set to a temperature at which the polymer has sufficient fluidity. The temperature may vary depending on a type of the polymer and may be set to, for example, 35° C. to 300° C. without a particular limitation.

The fluidity of the polymer solution 122 heated through the heating process increases, so that the polymer solution 122 flows into the gap 111 between the adjacent struts 10 by virtue of a capillary phenomenon. As a result, the curved portion 121 is formed on a surface of the polymer solution 122 in the inner lumen side of the stent so that the inner surface of the polymer solution 122 (polymer portions 120) exhibits a concave shape. According to this embodiment, the polymer solution 122 is filled in the gap 111 until end portions 123 of the curved portion 121 are closer to the stent lumen-side surface 112 of the struts 110. That is, according to one embodiment, the polymer solution 122 is filled in the gap 111 until the end portions 123 of the curved portion 121 are closer to the stent lumen-side surface 112 of the struts 110 than to the opposite surface of the struts 110. In this manner, since the polymer solution 122 is filled in the inside of the gap 111 as much as possible, a contact area between the polymer solution 122 and the struts 110, and further, a contact area between the polymer portions 120 and the struts 110 increases. Therefore, it is possible to improve fracture resistance on such an interface. In addition, since more polymer solution 122 is filled, a volume of the polymer portions 120 increases. Therefore, it is possible to improve a strength of the polymer portions 120 of itself. After the polymer solution 122 is filled in the gap 111, the polymer solution 122 is solidified to form the polymer portion 120. At the boundary lines L1, L2, the thickness of the polymer material may be at least equal to (no less than) the thickness of the struts or metal portions 110.

The process or method described above for forming the polymer portion 120 positioned in the gap 111 between two adjacent struts 110 to connect the adjacent struts 110 is preferably applied to the formation of all of the polymer portions 120 in the stent 100. The description above and below about the polymer portion 120 applies equally to all of the polymer portions 120.

Next, functional effects of the above-described stent and manufacturing method will be described.

According to this embodiment, compared to the struts 110 formed of metal, the relatively easily stretchable polymer portions 120 are thinned by forming the curved portion 121. Therefore, the polymer portions 120 are more easily stretchable. For this reason, for example, when a tensile force is applied tending to separate the struts 110 and the polymer portion 120 from each other at one or more of the connection regions by expanding the stent 100, it is possible to prevent a strong force from being easily applied to the interface between the struts (adjacent struts)110 and the polymer portion 120 by virtue of the stretching ability of the polymer portion 120. This thus improve fracture resistance.

In this embodiment, the peak P1 of the curved portion 121 (the thinnest portion of the polymer portion 120) is positioned in the center between two boundary lines L1 and L2 formed between the polymer portion 120 and the adjacent struts 110. In this configuration, the polymer portion 120 relatively easily stretches evenly between one side and the other side of the two adjacent struts 110, and a force is substantially uniformly applied to the two interfaces between the polymer portion 120 and both of the adjacent struts 110. For this reason, it is possible to prevent fracture resistance from being lowered by biasedly applying a stronger force to any one of the two interfaces.

Unlike this embodiment, a polymer portion having a contour line, for example, as indicated by the line L3 of FIG. 2 is not thinned by the curved portion 121 compared to the polymer portion 120 of this embodiment. That is, if the contour of the polymer portion 120 followed the line L3, the polymer portion 120 would not include a thinned portion. A polymer portion having a contour that follows the line L3 is not easily stretched relative to the struts 110.

Meanwhile, according to this embodiment, the peak P1 of the curved portion 121 is deviated (recessed) to the outer side of the stent from the line L3. As a result, the polymer portion 120 is thinned and becomes relatively easily stretchable. Therefore, an excessively strong force is not easily applied to the interface between the strut 110 and the polymer portion 120. In addition, it is possible to improve fracture resistance.

The invention is not limited to the aforementioned embodiments, and may be modified in various forms within the scope of the claims.

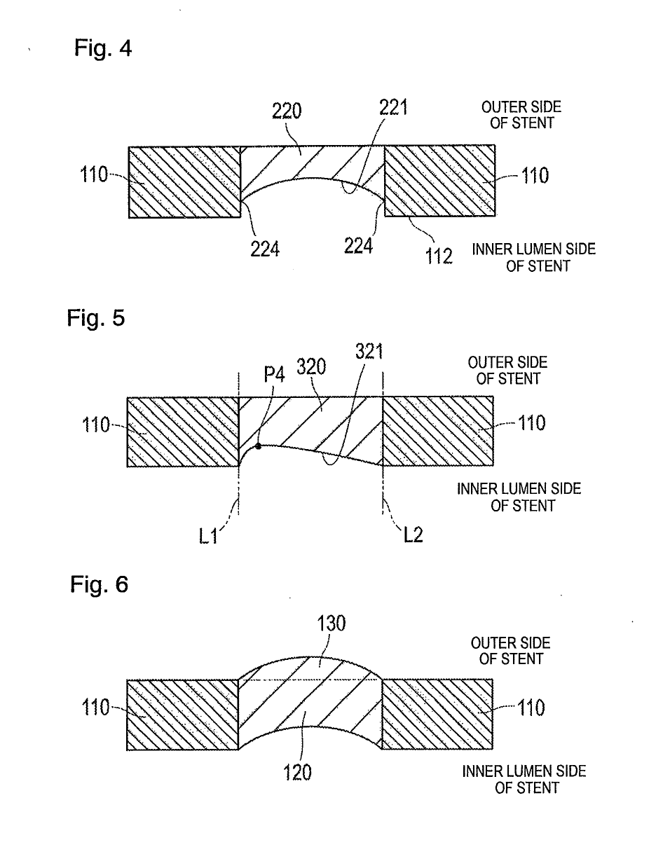

For example, as the polymer portion 220 illustrated in FIG. 4, the end portions 224 of the curved portion 221 may be spaced, in the direction toward the outer side of the stent, from the stent lumen-side surface 112 of the struts 110 so that the end portions 224 of the curved portion 221 do not reach or intersect the stent lumen-side surface 112 of the struts 110.

Another version of a polymer portion 320 is illustrated in FIG. 5. In the polymer portion 320, the peak P4 of the curved portion 321 (i.e., the most-thinned portion) is deviated from the center between the boundary lines L1 and L2. That is, the peak P4 is located at a position different from or spaced from the center between the boundary lines L1 and L2. In this case, the stretch of the polymer portion 320 is different between one side and the other side of the two adjacent struts 110, so that forces having different strengths are applied to the two interfaces between the polymer portion 320 and the two adjacent struts 110. For this reason, a relatively stronger force is intentionally applied to one of the two interfaces by deviating or moving the peak P4 away from the center. As a result, even when a fracture occurs, it is possible to control where the fracture occurs out of the two interfaces. In this embodiment, the end portions of the curved portion 321 may reach or intersect the stent lumen-side surface of the struts 110 as shown in FIG. 5.

In addition, as illustrated in FIG. 6, a polymer layer 130 may be formed outward of the polymer portion 120 of the stent. The polymer material may thus extend outwardly beyond the outwardly facing surfaces of the two adjacent struts 110 connected by the polymer material (i.e., the polymer layer 130 projects beyond the plane containing the outer surfaces of the two struts 110 as seen in FIG. 6). The polymer layer 130 is formed to match a position of the polymer portion 120 and is interspersed on the outer periphery of the stent 100. Since the polymer portion 120 is reinforced by the polymer layer 130, it is possible to improve a strength of the polymer portion 120 itself.

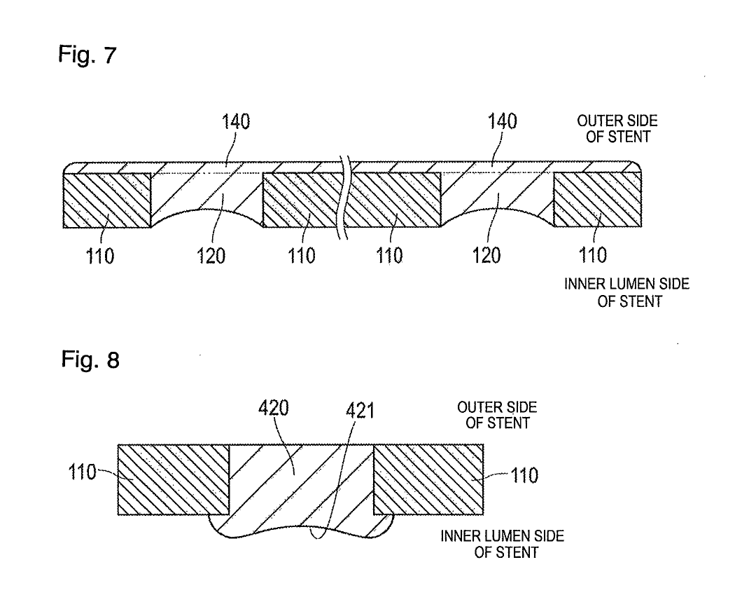

In addition, as illustrated in FIG. 7, a polymer layer 140 may be formed to continuously extend along the surface of the struts 110. The polymer layer 140 connects one of the polymer portions 120 and at least one of the other polymer portions 120. Since the polymer layer 140 reinforces the struts 110 and the polymer portion 120 across a wide range, it is possible to further improve the strength of the stent 100. The polymer layer 140 is preferably formed on or extends along the entire outer surface of the underlying struts 110. Alternatively, without being limited thereto, the polymer layer 140 may be partially formed to extend along a part of the outer surface of the struts 110. Furthermore, the polymer layer 140 may be formed inward of the stent 100. That is, the polymer layer 140 may be positioned on the inner side of the struts 110 (i.e., the bottom side of the struts in FIG. 7).

The polymer layers 130 and 140 are, for example, drug layers, but are not limited in this regard. That is, the polymer layers 130, 140 may contain a drug. In addition, the polymer layers 130 and 140 may be formed of the same material as that of the polymer portion 120 or a material different from that of the polymer portion 120. The polymer layers 130 and 140 are formed, for example, by further applying the polymer solution after formation of the polymer portion 120 and heating the further applied polymer solution for drying. A primer layer may also be formed before formation of the polymer layers 130 and 140.

FIG. 8 shows another variation in which a polymer portion 420 protrudes toward the stent inner lumen side with respect to the struts 110. That is, the polymer portion 420 extends inwardly beyond the inner surface of the adjacent struts 110 (i.e., the polymer portion 420 extends inwardly beyond the plane in which the inner surfaces of each of the two struts 110 lie). As a result, a volume of the polymer portion 420 increases. Therefore, it is possible to improve the strength of the polymer portion 420 of itself. As illustrated in FIG. 8, the inwardly facing side of the protruding polymer portion 420 includes the curved portion 421. Also, the polymer portion 420 may protrude to the inner side such that the polymer portion is in contact with the inwardly facing side of the metal portions or struts 110 as shown in FIG. 8.

In the polymer application process (polymer placement process) of the aforementioned embodiments, the polymer is placed in the gaps 111 by applying the polymer solution 122. However, the invention is not limited in this regard. For example, the polymer may be placed in the gap 111 by overlaying a solid sheet formed of polymer on the gap 111. In this case, the sheet is heated through a heating process and is molten, so that the molten polymer flows into the gap 111.

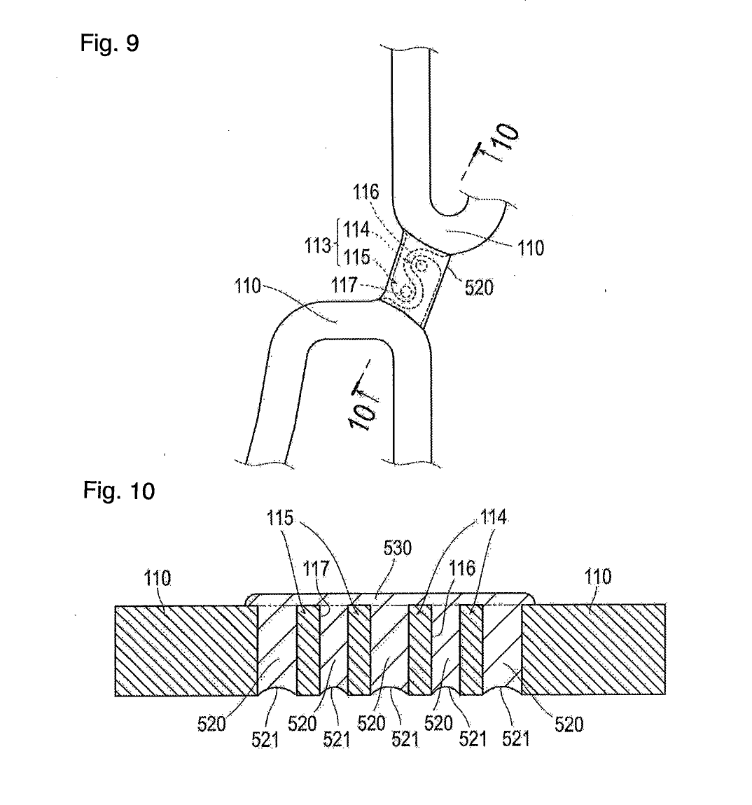

Another variation is illustrated in FIG. 9, Here, the stent is provided with a connection portion 113 along with the polymer portion 520. The polymer portion 520 and the connection portion 113 connect the struts 110 to each other.

The connection portion 113, which may be embedded in the polymer portion, includes a first connection portion 114 and a second connection portion 115. The first connection portion 114 is formed integrally with one of the two struts 110 connected to each other, and the second connection portion 115 is formed integrally with the other strut 110. The first and second connection portions 114 and 115 are formed of the same metal as that of the struts 110. The first and second connection portions 114 and 115 are configured to form a gap having a substantially S-shape therebetween. The first and second connection portions 114 and 115 may partially make contact with each other.

The first connection portion 114 is provided with a first through-hole 116, and the second connection portion 115 is provided with a second through-hole 117. The first and second through-holes 116 and 117 penetrate in a thickness direction (in a direction perpendicular to the plane of FIG. 9).

The first and second connection portions 114 and 115 are hook-shaped as shown in FIG. 9 and are caught with each other (axially and laterally overlap one another) when the struts 110 are separated from each other, so that connection between the struts 110 is maintained. For this reason, compared to a case where only the polymer portion 520 is provided, it is possible to more easily maintain a strength of the stent.

As illustrated in FIG. 10, the polymer portion 520 is formed in a gap between the struts 110 and the first connection portion 114, in a gap between the first connection portion 114 and the second connection portion 115, and in a gap between the second connection portion 115 and the struts 110. The polymer portions 520 formed in these gaps have curved portions 521 that are concave toward the outer side from the inner side of the stent. In addition, the polymer portions 520 are also formed in the first and second through-holes 116 and 117 and also have the curved portions 521. By virtue of the curved portions 521, it is possible to obtain the same functional effects as those of the curved portions 121 of the aforementioned embodiment.

The surfaces of the first and second connection portions 114 and 115 are covered by the polymer layer 530. The polymer layer 530 and the polymer portion 520 are formed integrally with each other. The first and second connection portions 114 and 115 are bonded to and supported by the polymer layer 530 and the polymer portion 520. Therefore, the first and second connection portions 114 and 115 are not easily removed.

The detailed description above describes embodiments of a catheter and operational method representing examples of the inventive catheter and operation disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
metal portions that together form a tubular frame possessing an outer periphery, the tubular frame including a gap extending through the tubular frame and at which two of the metal portions are positioned adjacent one another in a spaced-apart manner;
a polymer portion located in the gap and connecting the two metal portions to each other; and
the polymer portion including a curved portion that is curved and possesses a concave shape that is recessed toward an outer side of the stent from an inner side of the stent in a radial direction of the outer periphery.

2. The stent according to claim 1, wherein the polymer portion is connected to one of the two metal portions at one boundary line and the polymer portion is connected to the other of the two metal portions at an other boundary line, a peak at which the curved shape is deepest being positioned centrally between the one boundary line and the other boundary line as seen in a cross-section taken along a separation direction between the metal portions adjacent to the polymer portion.

3. The stent according to claim 1, wherein the polymer portion is connected to one of the two metal portions at one boundary line and the polymer portion is connected to the other of the two metal portions at an other boundary line, a peak at which the curved shape is deepest is positioned at a location different from a center between the one boundary line and the other boundary line as seen in a cross-section taken along a separation direction between the metal portions adjacent to the polymer portion.

4. The stent according to claim 1, wherein the polymer portion is connected to one of the two metal portions at one boundary line and the polymer portion is connected to the other of the two metal portions at an other boundary line, the curved shape of the curved portion intersecting the one boundary line at a first point and intersecting the other boundary line at a second point, a peak at which the curved shape of the curved portion is deepest being positioned in a location different from a straight line connecting the first and second points as seen in a cross section taken along a separation direction between the metal portions adjacent to the polymer portion.

5. The stent according to claim 1, wherein the gap is a first gap and the polymer portion is a first polymer portion, the two metal portions being two first metal portions;
further comprising a second gap extending through the tubular frame and at which two second metal portions are positioned adjacent one another in a spaced-apart manner;
further comprising a second polymer portion located in the second gap and connecting the two second metal portions to each other;
the second polymer portion including a curved portion that is curved and possesses a concave shape that is recessed toward the outer side of the frame from the inner side of the frame in the radial direction of the outer periphery;
a polymer layer formed on outwardly facing surfaces of the two first metal portions and the two second metal portions; and
the polymer layer connecting the first polymer portion and the second polymer portion.

6. The stent according to claim 1, further comprising a polymer layer on the polymer portion, the polymer layer projecting outwardly beyond a plane in which lies an outer surface of the two metal portions.

7. The stent according to claim 1, wherein the polymer portion projects inwardly beyond inner surfaces of the two metal portions so that the polymer portion projects inwardly beyond a plane in which lies the inner surface of each of the two metal portions.

8. The stent according to claim 1, further comprising first and second connection portions embedded in the polymer portion, the first connection portion being integrally formed with one of the two metal portions, and the second connection portion being integrally formed with the other of the two metal portions.

9. A stent comprising:
metal portions that together form a tubular frame possessing an outer periphery, the tubular frame including a gap extending through the tubular frame and at which two of the metal portions are positioned adjacent one another in a spaced-apart manner;
a polymer portion formed of biodegradable polymer and connecting the two metal portions to each other;
the polymer portion possessing an inwardly facing side facing towards an interior of the frame, the inwardly facing side of the polymer portion being curved.

10. The stent according to claim 9, wherein the polymer portion is connected to one of the two metal portions at one boundary line and the polymer portion is connected to the other of the two metal portions at an other boundary line, the curved inwardly facing side of the polymer portion including a point at which the curved inner side is deepest, the point at which the curved inwardly facing side of the polymer portion is deepest being positioned centrally between the one boundary line and the other boundary line as seen in a cross-section taken along a separation direction between the metal portions adjacent to the polymer portion.

11. The stent according to claim 9, wherein the polymer portion is connected to one of the two metal portions at one boundary line and the polymer portion is connected to the other of the two metal portions at an other boundary line, the curved inwardly facing side of the polymer portion including a point at which the curved inwardly facing side is deepest, the point at which the curved inwardly facing side of the polymer portion is deepest being positioned at a location different from a center between the one boundary line and the other boundary line as seen in a cross-section taken along a separation direction between the metal portions adjacent to the polymer portion.

12. The stent according to claim 9, wherein the polymer portion is connected to one of the two metal portions at one boundary line and the polymer portion is connected to the other of the two metal portions at an other boundary line, the curved inwardly facing side of the polymer including a point at which the curved inwardly facing side is deepest, the point at which the curved inwardly facing side of the polymer portion is deepest being positioned at a location spaced from a straight line connecting the first and second points as seen in a cross section taken along a separation direction between the metal portions adjacent to the polymer portion, the point at which the curved inwardly facing side of the polymer portion is deepest spaced from the straight line in a direction toward the outer periphery of the frame.

13. The stent according to claim 9, wherein the gap is a first gap, the polymer portion is a first polymer portion, and the two metal portions are two first metal portions;
further comprising a second gap extending through the tubular frame and at which two second metal portions are positioned adjacent one another in a spaced-apart manner;
further comprising a second polymer portion connecting the two second metal portions to each other;
the second polymer portion possessing an inwardly facing side facing towards the interior of the frame, the inwardly facing side of the polymer portion being curved
a polymer layer formed on outwardly facing surfaces of the two first metal portions and the two second metal portions; and
the polymer layer connecting the first polymer portion and the second polymer portion.

14. The stent according to claim 9, further comprising a polymer layer on the polymer portion, the polymer layer projecting outwardly beyond a plane in which lies an outer surface of the two metal portions.

15. The stent according to claim 9, wherein the polymer portion projects inwardly beyond inner surfaces of the two metal portions so that the polymer portion projects inwardly beyond a plane in which lies the inner surface of each of the two metal portions.

16. A method of manufacturing a stent, comprising:
placing polymer in contact with two metal portions of a tubular stent frame that possesses an outer periphery, the two metal portions being spaced apart from one another so that a gap exists between the two metal portions;
heating the polymer after placing the polymer in contact with the two metal portions to connect together the two metal portions by way of the polymer portion; and
the heating comprising heating the polymer so that the polymer is molten and flows to the gap to form a polymer portion that connects the two metal portions and includes an inwardly facing curved portion that is curved and possesses a concave shape that is recessed toward an outer side of the stent from an inner side of the stent in a radial direction of the outer periphery;.

17. The method according to claim 16, wherein the tubular stent frame comprises a plurality of gaps that extend through the stent frame and at each of which is located two metal portions that are spaced apart from one another, the placing of the polymer in contact with the two metal portions of the tubular stent frame comprising placing the polymer in contact with the two metal portions at a plurality of the gaps to connect together the two metal portions in each gap.

18. The method according to claim 17, wherein the placing of the polymer in contact with the two metal portions includes placing the polymer so that after the heating of the polymer portion of a tubular stent frame that possesses an outer periphery, the two metal portions being spaced apart from one another so that a gap exists between the two metal portions, and further comprising applying a polymer layer that contacts an outer surface of plural polymer portions as well as an outer surface of a plurality of the metal portions.

19. The method according to claim 16, wherein the polymer portion is connected to one of the two metal portions at one boundary line and the polymer portion is connected to the other of the two metal portions at an other boundary line, the placing of the polymer and the heating of the polymer being performed so that after the heating of the polymer, the inwardly facing curved portion of the polymer portion includes a point at which the curved portion is deepest and the point at which the curved portion is deepest being positioned at a center between the one boundary line and the other boundary line as seen in a cross-section taken along a separation direction between the metal portions adjacent to the polymer portion.

20. The method according to claim 16, wherein the polymer portion is connected to one of the two metal portions at one boundary line and the polymer portion is connected to the other of the two metal portions at an other boundary line, the placing of the polymer and the heating of the polymer being performed so that after the heating of the polymer, the inwardly facing curved portion of the polymer portion includes a point at which the curved portion is deepest and the point at which the curved portion is deepest being spaced from a center between the one boundary line and the other boundary line as seen in a cross-section taken along a separation direction between the metal portions adjacent to the polymer portion.
